(19) United States
(12) Patent Application Publication
(10) Pub. No.: US 2008/0108844 A1
(43) Pub. Date: May 8, 2008

(54) Non Hydroquinone Cyanoacrylate Cracking Process

(76) Inventor: John Baggaley, Oakham (GB)

Correspondence Address: ALTIMATIA, L.L.C., 16 ELM RIDGE ROAD, PENNINGTON, NJ, 08534, US

(21) Appl. No.: 11/854,639

(22) Filed: Sep. 13, 2007

(30) Foreign Application Priority Data
Nov. 2, 2006 (GB) GB062198.1

(51) Int. Cl. C07C 253/30; C07C 255/40

(52) U.S. Cl. 558/357; 558/441

(57) ABSTRACT

A process for producing a monomer comprising thermally cracking a pre-polymer in the presence of a stabiliser to form a monomer as a distillate , the stabiliser preventing the re-polymerisation of the monomer during cracking while not being carried over in the distillate in normally detectable amounts.

### CROSS REFERENCE TO RELATED APPLICATIONS

This application claims the benefit of GB Patent application number GBO62198.1-Filed 2nd November 2006.

Process for Producing a Monomer

The present invention relates to a process for producing a monomer particularly a monomer of a cyanoacrylate.

The process is especially suitable for producing a monomer for application as an adhesive particularly for medical applications e.g. sealing wounds and joining broken bones without the need for external mechanical appliances such as sutures.

The process is also particularly suitable for producing an adhesive for cosmetic applications e.g. for the decoration of finger and toenails.

Generally speaking these adhesives are made using an alkyl cyanoacrylate pre-polymer which itself is prepared by reacting formaldehyde with a cyanoacetate which may be methyl, ethyl or other homologue in the presence of a base such as piperidine, water then being removed to provide a pre-polymer of a cyanoacrylate.

In order to produce the monomer the cyanoacrylate pre-polymer is cracked by a thermolytic distillation process and the monomer is distilled over to a receiving vessel.

In order for the crack to be effective, acids in the form of known anionic stabilisers to stop anionic re-polymerisation during the formation of the monomer are added to the pre-polymer. In addition known radical stabilisers (also known as anti-oxidants) are also added to the pre-polymer to stop radical re-polymerisation of the monomer during the cracking process.

The crude cyanoacrylate monomer is then re-distilled and stabilised with anionic acid and radical stabilisers to prevent the monomer re-polymerising i.e. solidifying during storage and before use.

One problem with the current radical stabilisers used in the thermolytic process is that they tend to be carried over in the distillate. This is undesirable as their excessive loss from the polymer solution during the cracking process leaves it depleted in the particular stabiliser and therefore unstable. The polymer solution forms degradation products with associated reduced yields of the monomer. This stabilizer carry over phenomenon also restricts the temperature and vacuum at which the cracking and distillation can be undertaken and reduces process efficiency.

Another problem is that the currently preferred radical stabilizers for the cracking process are usually potentially harmful compounds such as hydroquinones or closely related alkylphenols with hydoquinone impurities and it is undesirable that they are carried over and present in significant amounts in the distilled monomer and thereafter in derived adhesive formulations, especially when these are to be used on the human body for say medical and/or cosmetic applications. These adhesive formulations in turn require stabilizers, including radical stabilizers, to provide adequate shelf-life but at the same time not unduly compromising adhesive curing performance which is affected by radical stabilzers. The currently preferred radical stabilizers for the cracking process are not necessarily the optimum stabilizers for the derived adhesive products and their presence compromises adhesive curing performance.

It is therefore an object of the present invention to provide a process whereby there is no carry over of the stabilisers in the thermolytic process or any such carry over of such stabilisers in monomers is held below the level where any toxins are present in potentially harmful amounts and, in addition, allow adhesive products to be subsequently formulated with selected suitable stabilizers to provide optimum shelf life stability and curing performance.

According therefore to the present invention, a process for producing a monomer comprises thermally cracking a pre-polymer in the presence of a stabiliser to form a monomer as a distillate, the stabiliser preventing the re-polymerisation of the monomer during cracking while not being carried over in the distillate in normally detectable amounts.

The thermal cracking process may take place under a vacuum.

Preferably the stabiliser has a vapour pressure which is such that at the temperature at which the pre-polymer cracks and the monomer distillate is formed, the stabiliser is not carried over to the distillate during the cracking process in normally detectable amounts.

Suitably the stabiliser has a vapour pressure which is lower than the boiling point of the prepolymer so that the stabiliser is not carried over to the distillate during the cracking process in normally detectable amounts.

Conveniently the stabiliser has a molecular weight which is sufficiently high such that the stabiliser is substantially not carried over to the distillate during the cracking process in normally detectable amounts.

The stabiliser which is present during thermal cracking may be a sterically hindered polyphenolic compound.

In one embodiment the stabiliser is selected from 1,3,5-trimethyl-2,4,6-tris(3,5-di-tert-butyl-4-hydroxybenzyl) benzene available as proprietary brands Anox 330 (Trade Mark) or Irganox 1330 (Trade Mark).

In another embodiment the stabiliser is hexaphenol.

In yet another embodiment the stabiliser is 1,1 Bis (2-methyl-4-tert-butylphenyl) butane available as the proprietary brand Lowinox 44B25 (Trade Mark).

In a still further embodiment the stabiliser is 1,1,3-Tris (2-methyl-4-hydroxy-5-t-butylphenyl) butane available as a proprietary brand Lowinox CA 22 (Trade Mark).

Preferably a stabiliser is added to the monomer so produced as the distillate to prevent the monomer reverting to the pre-polymer.

Suitably the selected stabiliser is a radical stabiliser which is added in the exact required amount.

Conveniently the radical stabiliser is selected from quinone, hydroquinone, p-tert-butyl catechol, p-methoxy phenol, 2,6-di-tert-butyl-p-cresol and 2,2-methylene-bis-(4-methyl-6-tert-butyl) phenol but this is by no means an exhaustive list and other unmentioned radical stabilisers may well be suitable and effective.

The stabiliser to be added to the monomer may be an anionic stabiliser which again can be added in the exact desired quantity.

In this case the anionic stabiliser is selected from methane sulphonic acid, p-toluene sulphonic acid, trifluoromethane sulphonic acid, hydroxy propane, sulphonic acid, sulphur dioxide and hydrofluoric acid but here again this is by no means an exhaustive list and other unmentioned anionic stabilisers may well be suitable and effective.

Suitably the pre-polymer is a cyanoacrylate compound which may be an alkyl, alkoxy, cycloaliphatic, unsaturated alkyl or aromatic cyanoacrylate.

Conveniently the cyanoacrylate is selected from a methyl, ethyl, n-propyl, iso propyl, n-butyl, iso-butyl, n-pentyl, 2-pentyl, 3-pentyl, n-hexyl, 2-hexyl, 3-hmay well be suitable hexyl, n-heptyl, 2-heptyl, 3-heptyl, 4-heptyl, n-octyl, 2-octyl, 3-octyl, methoxy ethyl, ethoxy ethyl, propoxy propyl, cyclobutyl, cycloheptyl, cyclohexyl, cyclooctyl, cyclopentyl and allyl cyanoacrylate but as before this is by no means an exhaustive list and other unmentioned cyanoacrylates may well be suitable and effective.

In one embodiment of the invention the cyanoacrylate pre-polymer is made from the reaction of a cyanoacetate and formaldehyde.

Preferably piperidine is added to the mixture to act as a catalyst.

Suitably the formaldehyde is paraformaldehyde.

Heptane may be added to the mixture to act as an azeotrope solvent but the addition of heptane is not essential and the reaction to form the prepolymer can take place in a mixture which is solvent-free.

An embodiment of the invention will now be particularly described with reference to the following example:

In this embodiment the pre-polymer is manufactured immediately prior to its being cracked to form the monomer. It will be appreciated however that it would be possible to crack the pre-polymer from an already prepared pre-polymer bought off the shelf so to speak.

The prepolymer to be manufactured in this example is ethyl cyanoacrylate. This is prepared from ethyl cyanoacetate and paraformaldehyde as the basic constituents.

The equipment described hereinafter in both the preparation of the pre-polymer and in the cracking process is conventional and well known in the art and so will not be described in detail.

Firstly, in order to prepare the prepolymer, all the compounds to form it are added to a conventional three necked 500 ml borosilicate glass mixing vessel fitted with a stirrer. There is also provided a known type Dean and Stark condenser connected to an outlet end of the mixing vessel together with a monomer receiving vessel for receiving the monomer condensate. The receiving vessel is connected to an outlet end of the condenser, a thermometer being provided to measure the temperature of the contents of the glass mixing vessel and a heating mantle to heat the contents of the glass vessel.

To the glass mixing vessel are added 241 gm of ethyl cyanoacetate, 0.7 gm piperidine, 60 gm of paraformaldehyde and 70 gm heptane which takes no part in any reaction but merely acts as a solvent. The cyanoacetate, piperidine and paraformaldehyde are reacted together under reflux conditions with any water forming in the vessel as a result of the reaction being removed by azeotropic distillation via the Dean and Stark condenser as is conventional.

Secondly, in order to prepare the monomer from the prepolymer just produced iin the mixing vessel, 6.9 gm of phosphorus pentoxide, 0.6 gm p-toluene sulphonic acid and 4.6 gm of Anox 330 are added to the prepolymer in the mixing vessel. The glass mixing vessel is then heated by the mantle so that the prepolymer is allowed thermally to depolymerise or crack at a temperature of between 140 and 180° C. under a vacuum of 7×105 Pa so as to vaporise and form the monomer which distils over to the receiving vessel via the condenser.

Analysis of the monomer so produced shows that it contains less than 1 ppm of the Anox 330. This is the analytical limit of detection.

The stabiliser used in the preparation of the monomer (in the case described Anox 330) will have a vapour pressure which is lower than the boiling point of the ethyl cyanoacrylate prepolymer so that the stabiliser is not distilled over to the receiving vessel with the monomer.

Although the stabiliser described is Anox 330, Irganox 1330 is also suitable and indeed any suitable sterically hindered phenolic compound would suffice. Usually such a compound will have a molecular weight sufficiently high such that it is not carried over with the monomer distillate. A compound with a molecular weight of between 300 and 900 is generally suitable.

A crude monomer is formed from the cracking process and optionally a second distillation is made using Anox 330 again to purify the monomer.

In a third step, after the monomer has been prepared, as is conventional, stabilisers of both the radical type and the anionic type are typically added to the receiving vessel and will dissolve in the condensate carried over from the glass mixing vessel. These stabilisers (also known as inhibitors) will serve to prevent the monomer re-polymerising on storage. They can different to the stabiliser used in the cracking/distillation process. However the stabilisers used in the cracking/distillation process could be used as the stabilisers to stabilise the monomer so produced. Consequently, Anox 330 could be used as a radical stabiliser but it does not impart a very long shelf-life to the monomer so produced and so is not preferred as a stabiliser for the monomer.

The preferred stabilisers can and do have lower molecular weights and higher vapour pressures than the stabiliser e.g. Anox 330 used in the cracking process to form the monomer since these monomer stabilisers are not going to be used at high temperatures but only at room temperature. They are simpler, smaller compounds in structure than the stabiliser used in the cracking process and are therefore generally far cheaper.

Typical radical stabilisers or inhibitors are quinone, hydroquinone, p-tert-butyl catechol, p-methoxy phenol, 2,6-di-tert-butyl-p-cresol and 2,2-methllene-bis-(4-methyl-6-tert-butyl) phenol. These inhibitors are typically added in amounts sufficient to form concentrations of between 10 ppm and 10000 ppm in respect of the weight of the adhesive composition. Most preferably the concentration lies within the range of between 100 ppm and 1000 ppm.

Typical anionic polymerisation stabilisers are methanesulphonic acid, p-toluene sulphonic acid, trifluoromethane sulphonic acids, hydroxy propane sulphonic acid, sulphur dioxide and hydrofluoric acid. These stabilisers are typically added in amounts providing concentrations of between 5 ppm and 500 ppm with respect to the weight of the adhesive composition.

1. A process for producing a monomer comprising thermally cracking a pre-polymer in the presence of a stabiliser to form a monomer as a distillate, the stabiliser preventing the re-polymerisation of the monomer during cracking while not being carried over in the distillate in normally detectable amounts.

2. A process as claimed in claim 1 in which the thermal cracking process takes place under a vacuum.

3. A process as claimed in claim 1 or claim 2 in which the stabiliser has a vapour pressure which is such that at the temperature at which the prepolymer cracks and the monomer distillate is formed, the stabiliser is not carried over to the distillate during the cracking process in normally detectable amounts.

4. A process as claimed in claim any of claims 1 to 3 in which the stabiliser has a vapour pressure which is lower than the boiling point of the prepolymer so that the stabiliser is not carried over to the distillate during the cracking process in normally detectable amounts.

5. A process as claimed in any one of claims 1 to 4 in which the stabiliser has a molecular weight, which is sufficiently high such that the stabiliser is not carried over to the distillate during the cracking process in normally detectable amounts.

6. A process as claimed in any one of claims 1 to 5 in which the stabiliser is a sterically hindered polyphenolic compound.

7. A process as claimed in any one of claims 1 to 6 in which the stabiliser is selected from 1,3,5-trimethyl-2,4,6-tris (3,5-di-tert-butyl-4-hydroxybenzyl) benzene available as the proprietary brand Anox 330 (Trade Mark) and the proprietary brand Irganox 1330 (Trade Mark)

8. A process as claimed in any one of claims 1 to 6 in which the stabiliser is hexaphenol.

9. A process as claimed in any of claims 1 to 6 in which the stabiliser is 1,1 Bis (2-methyl-4-tert-butylphenyl) butane available as the proprietary brand Lowinox 44B25 (Trade Mark).

10. A process as claimed in any of clams 1 to 6 in which the stabiliser is 1,1,3-Tris (2-methyl-4-hydroxy-5-t-butylphenyl) butane available as the proprietary brand Lowinox CA 22 (Trade Mark).

11. A process as claimed in any of the preceding claims in which a stabiliser is added to the monomer so produced as the distillate to prevent the monomer reverting to the prepolymer.

12. A process as claimed in claim 11 in which the stabiliser is a radical stabiliser.

13. A process as claimed in claim 12 in which the stabiliser is selected from quinone, hydroquinone, p-tert-butyl catechol, p-methoxy phenol, 2,6-di-tert-butyl-p-cresol and 2,2-methylene-bis-(4-methyl-6-tert-butyl) phenol.

14. A process as claimed in any of the preceding claims in which the stabiliser is an anionic stabiliser.

15. A process as claimed in claim 14 in which the stabiliser is selected from methane sulphonic acid, p-toluene sulphonic acid, trifluoromethane sulphonic acid, hydroxy propane sulphonic acid, trifluoromethane sulphonic acid, hydroxy propane sulphonic acid, sulphur dioxide and hydrofluoric acid.

16. A process as claimed in any of the preceding claims in which the pre-polymer is a cyanoacrylate compound.

17. A process as claimed in claim 16 in which the prepolymer is an alkyl, alkoxy, cycloaliphatic, unsaturated alkyl or aromatic cyanoacrylate.

18. A process as claimed in claim 16 or claim 17 in which the cyanoacrylate is selected from a methyl, ethyl, n-propyl, iso propyl, n-butyl, iso butyl, n-pentyl, 3-pentyl, n-hexyl, 2-hexyl, 3-hexyl, n-heptyl, 2-heptyl, 3-heptyl, 4-heptyl, n-octyl, 2-octyl, 3-octyl, methoxy ethyl, ethoxy ethyl, propoxy propyl, cyclobutyl, cycloheptyl, cyclohexyl, cyclooctyl, cyclopentyl or allyl cyanoacrylate.

19. A process as claimed in any of claims 16 to 18 in which the pre-polymer is made from a reaction of a cyanoacetate and formaldehyde.

20. A process as claimed in 19 in which piperidine is added to the mixture.

21. A process as claimed in claim 19 or claim 20 in which the formaldehyde is paraformaldehyde.

22. A process as claimed in any of claims 19 to 21 in which heptane is added to the mixture.

23. A process substantially as hereinbefore described with reference to the example.
